(19) United States
(12) Patent Application Publication
(10) Pub. No.: US 2020/0188155 A1
(43) Pub. Date: Jun. 18, 2020

(54) KNEE BRACE

(71) Applicant: Bryan Balentine, Birmingham, AL (US)

(72) Inventor: Bryan Balentine, Birmingham, AL (US)

(21) Appl. No.: 16/224,711

(22) Filed: Dec. 18, 2018

(51) Int. Cl. A61F 5/01

(52) U.S. Cl. CPC A61F 5/0123; A61F 13/08; A61F 2005/0167; A61F 5/0109

(57) ABSTRACT

A knee brace includes a body and a handle. The handle comprises an attachment mechanism, such as a slot, to engage the knee brace. The handle may include a slot, or aperture, for receiving one or more components of the knee brace, such as a linkage. The knee brace, or components thereof, may be constructed of an elastic synthetic polymer, such as nylon.

### TECHNICAL FIELD

The present disclosure relates to a knee brace. More specifically, the disclosure is directed towards a knee brace that includes a body having at least two supports, each of the supports configured to circumferentially extend around a leg of a subject, a linkage longitudinally connecting the at least two supports, and a handle engageable with the linkage.

### BACKGROUND OF INVENTION

Knee braces, or knee orthoses, are commonly employed to resist excessive movement of the knee joint in humans. In particular, knee braces provide support to the patella and surrounding ligaments. Knee braces are used during physical activity, such as sports, recovering from an injury, and to prevent injury. In instances where a knee brace is used with subjects having diseases causing impairment of muscles surrounding the knee, the knee brace can prevent flexion or extension instability of the knee.

Because knee braces are generally desired to provide stability to the knee without excessively limiting range of motion in the subject, existing knee braces are not positionally secure, or do not allow the subject to adjust the position of the knee brace without excessively limiting range of motion in the subject. Thus, a need exists for knee braces that can be positionally adjusted or secured with the subject while the subject maintains a sufficient range of motion.

### BRIEF SUMMARY

In one aspect, a knee brace includes a body having at least two supports, each of the supports configured to circumferentially extend around a leg of a subject. The knee brace includes a link that longitudinally connects the at least two supports. The knee brace includes a handle that is engageable with the linkage. The handle may include an engagement portion including an aperture configured to slideably receive the linkage. The linkage may be adjustable in length between the at least two supports.

In a second aspect, a knee brace comprises a body having at least two supports, each of the supports configured to circumferentially extend around a leg of a subject a linkage longitudinally, securely, and removably connecting the at least two supports; and a handle including an aperture configured to cooperatively receive the linkage.

The linkage may include an internal aperture and the handle may comprise a boss configured to be cooperatively received by the internal aperture. The knee brace bay include a lock for releasably securing the handle with the linkage in a secured position. The linkage may include a linkage profile, and the aperture has an aperture profile, and the aperture profile is configured for the aperture cooperatively engage the linkage.

The at least two supports may include a first superior support extending from the linkage, and a first inferior support extending from the linkage in a direction generally opposite of the first superior support. The at least two supports may comprise a first superior support and a second superior support extending from the linkage, and a first inferior and a second inferior support extending from the linkage in a direction opposite of the first superior support and second superior support. The knee brace may include a third support configured to circumferentially extend around a leg of the subject.

The knee brace may include a hinge connecting the third support to one of the at least two supports around the leg of the subject.

### BRIEF DESCRIPTION OF DRAWINGS

It should be noted that identical features in different drawings are shown with the same reference numeral.

FIG. 1 shows a front perspective view of one embodiment of a knee brace.

FIG. 2 shows a front perspective view of another embodiment of a knee brace.

FIG. 3 shows a front perspective view of a yet another embodiment of a knee brace.

FIG. 4 shows a front perspective view of a yet another embodiment of a knee brace.

FIG. 5 shows a front perspective view of a further embodiment of a knee brace.

FIG. 6 shows a front perspective view of another embodiment of a knee brace.

FIG. 7 shows a front perspective view of a handle according to an embodiment of a knee brace.

### DETAILED DESCRIPTION

Reference now will be made in detail to the embodiments of the present disclosure. It will be apparent to those of ordinary skill in the art that various modifications and variations can be made to the teachings of the present disclosure without departing from the scope of the disclosure. For instance, features illustrated or described as part of one embodiment can be used with another embodiment to yield a further embodiment.

Thus, it is intended that the present disclosure covers such modifications and variations as come within the scope of the appended claims and their equivalents. Other objects, features, and aspects of the present disclosure are disclosed in or are apparent from the following description. It is to be understood by one of ordinary skill in the art that the present disclosure is a description of exemplary embodiments only and is not intended as limited the broader aspects of the present disclosure.

For the sake of clarity, not all reference numerals are necessarily present in each drawing Figure. In additional, positional terms such as “upper,” “lower,” “side,” “top,” “bottom,” “vertical,” “horizontal,” etc. refer to the knee brace when in the orientation shown in the drawings. The skilled artisan will recognize that the knee brace can assume different orientations when in use.

A knee brace has been developed. The knee brace of the present disclosure is intended to retain a position on a subject while still allowing the subject to have a comfortable range of motion while wearing the knee brace. The knee brace resists migration and/or can be easily adjusted by the subject to a desired position to continue to provide support in the desired area on the subject.

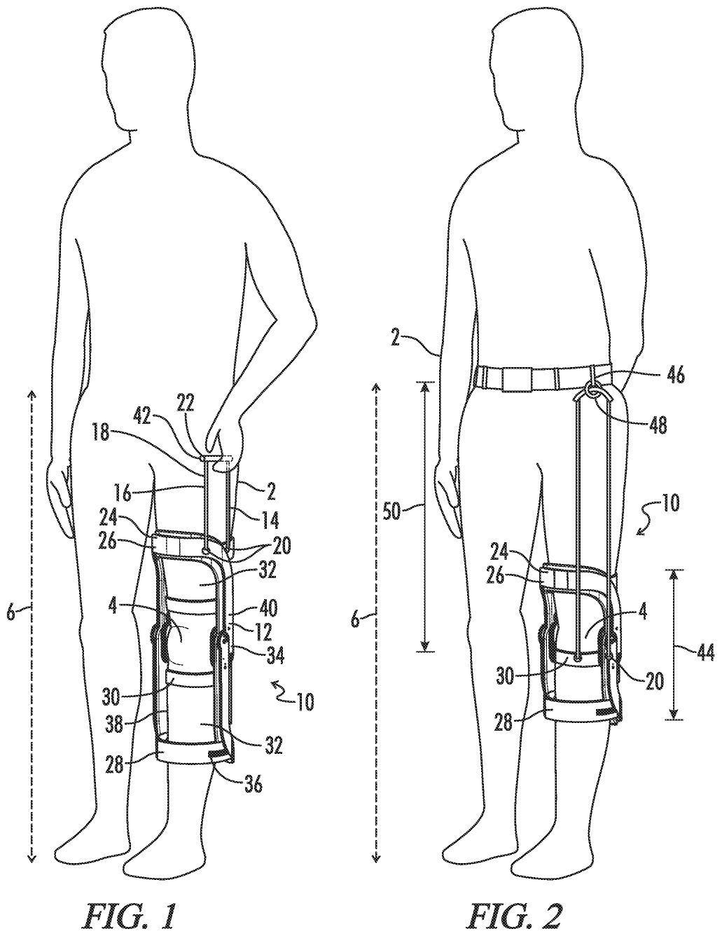

One embodiment of the knee brace is shown in FIG. 1. In this embodiment, a knee brace 10 includes a body 12 configured to circumferentially extend around a knee region 4 and/or tissue surrounding the knee region 4 of a subject 2. The body 12 may be constructed of, for example, any suitable material such as one or more metals, a polymer such as neoprene, an alloy, or any combination thereof. A strap 14 is provided on the body 12.

The strap 14 includes a body end 16 disposed toward the body 12 and a handle end 18 disposed oppositely to the body end 16 when the strap is in an extended position, as shown. The strap 14 may be flexible. The strap 14 may be constructed of any suitable material, such as a rigid polymer or an elastic flexible polymer such as nylon. The strap 14 may be releasably coupled, or attached, with the body 12 by at least one strap connection member 20. By way of example, the handle connection member 20 may be a hook and loop fastener, a snap, a zipper, a buckle, a clasp, a button, a grommet, or any combination thereof. A handle 22 is disposed at the handle end 18 of the strap 14 and secured to the body 12 by the strap 14. The handle 22 may be rigid or flexible, and the handle 22 may have a rigid member 42 disposed thereon. The handle 22 or rigid member 42 may include at least one gripping member (not shown) disposed thereon such that a user can conveniently and ergonomically grip the handle 22.

The knee brace 10 may include a plurality of supports 24, each plurality of support 24 configured to circumferentially extend around the knee region 4 and secured by a leg member 34 on the body 12. The leg member 34 may extend substantially parallel to a sagittal plane 6 of the subject 2. Alternatively, the knee brace 10 may have a single support 24 (not shown) configured to circumferentially extend around the knee region 4. The plurality of supports 24 may include a superior support 26 positioned at the upper end of the knee brace 10. The plurality of supports 24 may include an inferior support 28 positioned at the lower end of the knee brace 10. The plurality of supports 24 may include a center support 30 disposed between the superior support 26 and the inferior support 28. The plurality of supports 24 may define a plurality of apertures 32, each aperture disposed between two supports 24. Advantageously, in embodiments of the knee brace 10 having the plurality of supports 24 and plurality of apertures 32, the knee brace 10 is lightweight, provides support to the knee region 4, and is breathable for the subject 2. The plurality of supports 24 may be have an adjustable feature 36, such as a hook and loop fastener, to fit on the knee regions 8 of different sizes and ages of subjects 2.

The strap 14 may be positioned on and secured with the plurality of supports 24, such as the superior support 26, the inferior support 28, the center support 30 (as shown), or any combination thereof. Advantageously, the handle 22 and strap 14 may be configured and dimensioned such that when the handle 22 and the strap 14 are in the extended position, handle 22 is conveniently reachable for the subject 2, and when the handle 22 and the strap 14 are in a relaxed position, the handle 22 and the strap 14 do not reach the ground. This feature prevents the handle 22 from being a tripping hazard for the subject 2 while maintaining convenient access for the subject 2. The strap 14 may be constructed of any suitable material, such as a rigid polymer or an elastic flexible polymer such as nylon.

Problematically, conventional knee braces migrate, or slide, down the leg of the subject when they are in-use. When the conventional knee braces migrate, they are unable to provide adequate support to the associated knee region and become uncomfortable. However, in embodiments of the knee brace 10 having the strap 14 and the handle 22, the subject 2 can readjust (i.e., pull upwardly) on the brace 10 to restore the brace 10 to its desired position to maintain efficacy and comfort.

As shown in FIG. 1, the body 12 includes a medial portion 38 (i.e., the inner portion) and a lateral portion 40 (i.e., an oppositely outwardly facing portion) disposed opposite from the medial portion. The strap 14 may be secured to the lateral portion 40 of the body 12. Advantageously, in embodiments of the knee brace 10 having strap 14 secured to the lateral portion 40 of the body 12, the positioning of the strap 14 allows the subject 2 to conveniently reach the handle 22, as the handle 22 is in, or proximate to, a plane 6 running lengthwise to the subject's arm.

As shown in FIG. 2, an embodiment of the knee brace 10 has the body 12 configured to circumferentially extend around the knee region 4 of the subject 2. The body 12 includes a length 44 extending parallel to the plane 6. The plurality of supports 24 are configured and dimensioned to extend circumferentially around the knee region 4. The plurality of supports 24 include the superior support 26, the inferior support 28, and the center support 30 disposed within, or between, the superior support 26 and the inferior support 28. Each of the plurality of supports 24 is coupled with, or attached to, the leg member 34 at the at least one strap connection member 20. The leg member 34 extends substantially parallel to the sagittal plane 6. The flexible strap 14 is configured to vertically secure one or more of the plurality of supports 24, such as the superior support 26 or the center support 30 (as shown in FIG. 2) with a belt or a belt loop 46. The flexible strap 14 may releasably attach to the belt or belt loop 46 with a fastener 48, such as, by way of example, a loop, a hook, a button, a hook, or a self-locking clasp.

The strap 14 may be dimensioned to extend a length 50 of about 25% to 250% of the length 44 of the body 12. When the strap 14 is secured with belt or belt loop 46, the belt or belt loop 46 provides vertical support such as to prevent the knee brace 10 from migrating downwards. The strap 14 may be constructed of any suitable material, such as a rigid polymer or an elastic flexible polymer such as nylon.

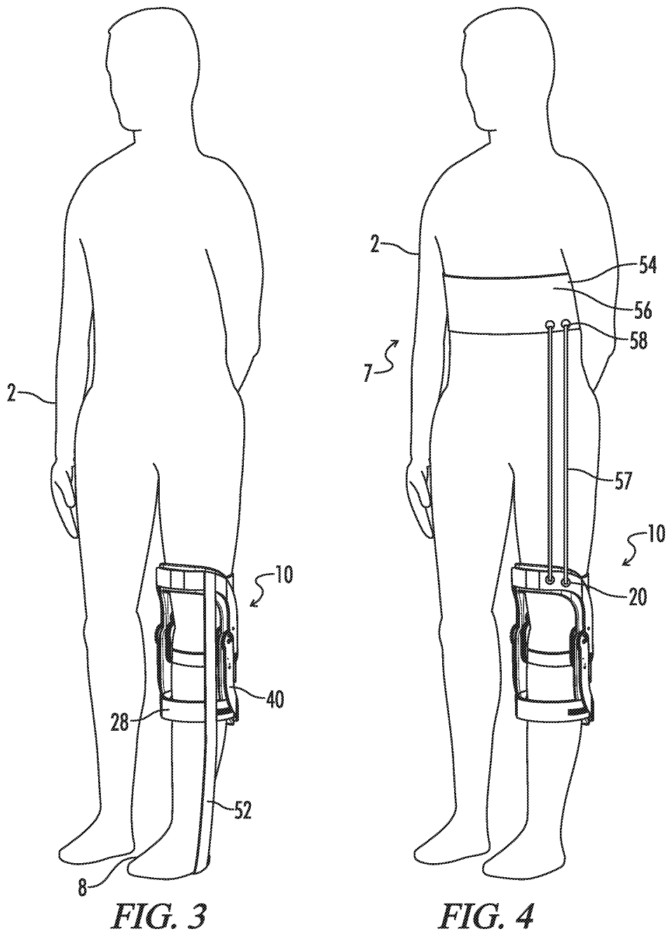

As illustrated in FIG. 3, an embodiment of the knee brace 10 includes the flexible strap 14 configured to vertically secure one or more of the plurality of supports 24, such as the inferior support 28, with a foot member 52. The foot member 52 may be configured and dimensioned to receive at least a portion of a foot 8 of the subject 2. For example, the foot member 52 may extend from the leg member 34 on both the lateral portion 40 and the medial portion 38 of the brace 10 such that the foot member 52 is dimensioned and configured to receive the heel area of the foot 8. The foot member 52 may extend from one or more of the plurality of supports 24. When the foot member 52 is in use, the foot member vertically secures the knee brace 10 to prevent migration, including vertical, lateral, and rotational migration.

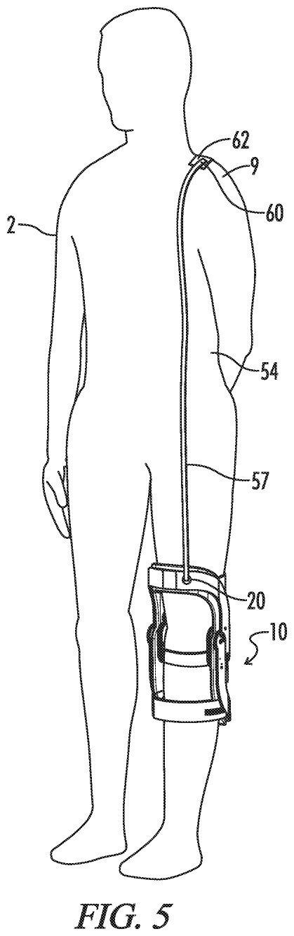

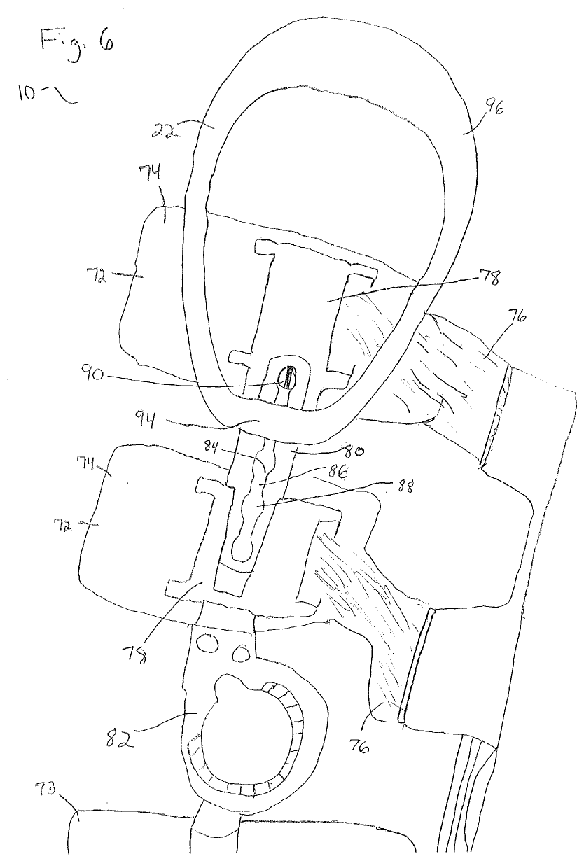

As shown in FIGS. 4 and 5, an embodiment of the knee brace 10 includes an upper member 54 configured to vertically secure the knee brace 10. The upper member 54 may attach to various points on the subject 2 above the waist. As illustrated in FIG. 4, the upper member 54 may comprise a torso member 56 that extends circumferentially on a torso 7 of the subject 2. The upper member 54 may include an additional flexible strap 57 that releasably couples with each of the knee brace 10 at the at least one strap connection member 20 and the upper member 54 at an upper member connection member 58, independently. Advantageously, in embodiments of the knee brace 10 having torso member 56, the knee brace is vertically secured, and forces from exerted from the knee brace 10 are distributed across torso member 56 for subject comfort. The upper member may attach to the knee brace 10 at the at least one strap connection member 20.

As illustrated in FIG. 5, the upper member 54 may comprise a shoulder member 60. Shoulder member 60 is configured and dimensioned to extend over a shoulder 9 of the subject 2. The shoulder member 60 may comprise a shoulder pad 62 so as to distribute weight and pressure across the shoulder 9 when the knee brace 10 is in use. The shoulder member 60 may extend from the knee brace 10 both on the dorsal and ventral side of the patient 2, providing vertical support to the knee brace 10 to maintain the knee brace 10 in a desired position. The shoulder member 60 may be configured to be worn on the shoulder 9 on the same side of the body on which the knee brace 10 is worn, or the shoulder member 60 may be configured to be worn on the shoulder 9 on the other side of the body.

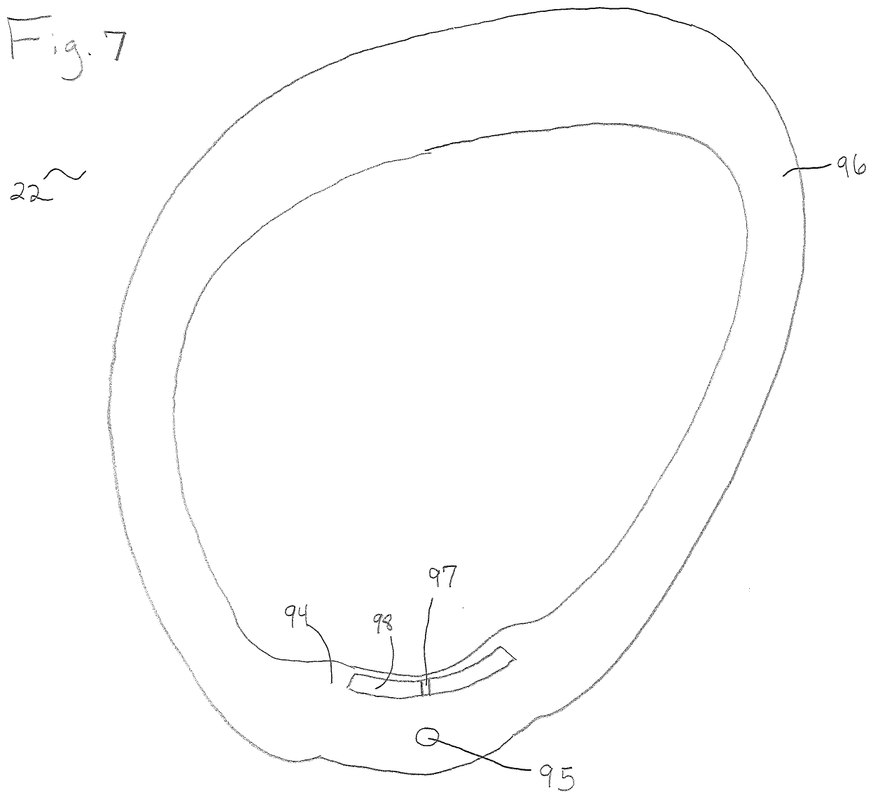

FIG. 6 depicts an alternate embodiment of the knee brace 10 having a handle 22. As shown in FIG. 6, at least one superior support 72 wraps circumferentially around the leg of the subject 2 (e.g., thigh above the knee), and at least one inferior support 73 wraps around the leg of subject 2 (e.g., below the knee). Each support 72, 73, as shown in FIG. 6, includes one or more rigid or semi-rigid curved portions 74 that engage the knee, thigh, and/or leg and which are connected to each other circumferentially by one or more straps 76. One curved portion 74 in each support may be generally dispositioned on the mediate side of the knee or leg, and the other curved portion 74 may be generally dispositioned on the lateral side of the knee or leg, when worn by the subject 2. Each curved portion 74 has an interface 78 for attaching to a linkage 80 such as that depicted in FIG. 6. The embodiment of the knee brace 10, as shown in FIG. 6, has four linkages 80. A first linkage 80 connects the curved portions 74 of the superior supports 72 on the mediate side of the knee. A second linkage connects the curved portions 74 of the superior supports 72 on the lateral side of the knee. A third linkage 80 connects the curved portions 74 of the inferior supports 73 on the mediate side of the knee (this linkage is not shown in FIG. 6). A fourth linkage connects the curved portions 74 of the inferior supports 73 on the lateral side of the knee. Between the superior supports 72 and the inferior supports 73 is a hinge 82, connected to both the superior support 72 and the inferior support 73 that are nearest to the hinge 82, that is generally dispositioned adjacent to the knee. There is a hinge 82 on the lateral side of the knee and one hinge 82 on the mediate side of the knee.

In the embodiment shown in FIG. 6, each linkage 80 has a profile having a capsular cross section, and each linkage 80 has an internal aperture 84. The internal aperture 84 is characterized by a plurality of openings (which may generally circular as shown) 88 that are joined by generally longitudinal openings 86. Each curved portion 74 is connected to the linkage 80 at the interface 78. In the embodiment depicted in FIG. 6, the connection is formed using a knob 90 that is placed on one side of the linkage 80 and connects to the interface 78 of the curved portion 74 that is placed on the opposite side of the linkage 80 through a connection made through the generally circular portion 88 of the linkage aperture 84. The connection may be by screwing, snapping on, interlocking elements or other secure mechanisms for connecting and disconnecting the knob 90 to the interface 78 on the curved portion 74.

Other alternative embodiments for the securing and connection of the curved portions 74 to the linkage 80 are possible. For example, the interface of the curved portion may have an adjustable receptacle that receives the linkage and can be placed at a desired length. Alternatively, the connector may have a hook or other protrusion onto which the linkage can be hung. Alternatively, the linkage may have an interlocking element that coordinates with an interlocking element on the connector. Other ways of connecting the linkage to the connector in a manner that allows disengagement are also possible.

In the embodiment shown in FIG. 6, the handle 22 has an engagement portion 94 that engages with the linkage 80, and a gripping portion 96 that rises upward towards the subject's hand. The engagement portion 94 includes an aperture 98, or opening, configured to cooperative receive the linkage 80. The aperture 98 and the linkage may have cooperatives profiles such that the aperture 98 may engage with the linkage 80 securely and slideably. The aperture 98 may slip, or slide, along the linkage 80. In this manner, the handle 22 can be engaged with the linkage 80 prior to the linkage 80 being connected to the curved portions 74 of the supports 72, 73. This results in the handle 22 being able to be installed with the brace 10, specifically linkage 80, when the interface 78 is disconnected from the linkage 80. Moreover, this results in the handle 22 adjustable along the length of the linkage 80, so the subject 2 can adjust the handle 22 to a desired height. In some embodiments, the engagement portion 94 and/or the linkage 80 may further include a lock 95, such as a clasp, snap, resiliently biased button, or other mechanism for locking onto the linkage 80 once the handle 22 is placed at a desired position. In another embodiment, the handle 22 may be connected to the hinge 82. Such a connection may be made in a manner similar to that described with reference to connecting to the supports above, and as analogously depicted in FIGS. 1-5.

FIG. 7 depicts an isolated perspective view of the handle 22 for use in the embodiment depicted in FIG. 6. The aperture 98 in the engagement portion 94 of the handle 22 is shown and, in this embodiment, is contoured to closely fit over the linkage 80 shown in FIG. 6. The handle 22 may comprise a boss 97 on the engagement portion 94, the boss 97 configured to be cooperatively received by the internal aperture 84 of the linkage 80. The profile of the boss 97 may be cooperative with the profile of the internal aperture 84, such that, for example, the boss 97 is slideably received in the internal aperture 84. In this way, the handle 22 may be adjustable height-wise, yet further resist rotation, especially in embodiments having linkages 80 that are round in profile.

Although embodiments of the disclosure have been described using specific terms, devices, and methods, such description is for illustrative purposes only. The words are words of description rather than of limitation. It is to be understood that changes and variations may be made by those of ordinary skill in the art without departing from the sprit or scope of the present disclosure, which is set forth in the following claims. It is further noted that any range provided herein provides support and a basis for any subset within that range. Further embodiments of the disclosure contain combinations, or exclusions, of different embodiments described herein.

Thus, although there have been described particular embodiments of the present invention of a new and useful knee brace, it is not intended that such references be construed as limitations upon the scope of this invention except as set forth in the following claims.

What is claimed is:

1. A knee brace, comprising:
a body having at least two supports, each of the supports configured to circumferentially extend around a leg of a subject;
a linkage longitudinally connecting the at least two supports; and
a handle engageable with the linkage.

2. The knee brace of claim 1, further comprising:
a third support configured to circumferentially extend around a leg of the subject; and
a hinge connecting the third support to one of the at least two supports around the leg of the subject.

3. The knee brace of claim 2, wherein the handle comprises an engagement portion including an aperture, the aperture configured to slideably receive the linkage.

4. The knee brace of claim 1, wherein the handle comprises an engagement portion including an aperture, the aperture configured to slideably receive the linkage.

5. The knee brace of claim 1, wherein the linkage is adjustable in length between the at least two supports.

6. The knee brace of claim 1, wherein the at least two supports comprise a first superior support extending from the linkage, and a first inferior support extending from the linkage in a direction generally opposite of the first superior support.

7. The knee brace of claim 1, wherein the at least two supports comprise a first superior support and a second superior support extending from the linkage, and a first inferior and a second inferior support extending from the linkage in a direction opposite of the first superior support and second superior support.

8. The knee brace of claim 1, wherein the linkage comprises an internal aperture, and wherein the handle comprises a boss configured to be cooperatively received by the internal aperture.

9. The knee brace of claim 1, wherein the knee brace comprises a lock for releasably securing the handle with the linkage in a secured position.

10. The knee brace of claim 1, wherein the linkage has a linkage profile, and the aperture has an aperture profile configured for the aperture cooperatively engage the linkage.

11. A knee brace, comprising:
a body having at least two supports, each of the supports configured to circumferentially extend around a leg of a subject;
a linkage longitudinally, securely, and removably connecting the at least two supports; and
a handle including an aperture configured to cooperatively receive the linkage.

12. The knee brace of claim 10, wherein the linkage has a linkage profile, and the aperture has an aperture profile, and the aperture profile is configured for the aperture cooperatively engage the linkage.

13. The knee brace of claim 10, further comprising:
a third support configured to circumferentially extend around a leg of the subject; and
a hinge connecting the third support to one of the at least two supports around the leg of the subject.

14. The knee brace of claim 12, wherein the handle comprises an engagement portion including an aperture, the aperture configured to slideably receive the linkage.

15. The knee brace of claim 10, wherein the handle comprises an engagement portion including an aperture, the aperture configured to slideably receive the linkage.

16. The knee brace of claim 10, wherein the linkage is adjustable in length between the at least two supports.

17. The knee brace of claim 10, wherein the at least two supports comprise a first superior support extending from the linkage, and a first inferior support extending from the linkage in a direction generally opposite of the first superior support.

18. The knee brace of claim 10, wherein the at least two supports comprise a first superior support and a second superior support extending from the linkage, and a first inferior and a second inferior support extending from the linkage in a direction opposite of the first superior support and second superior support.

19. The knee brace of claim 10, wherein the linkage comprises an internal aperture, and wherein the handle comprises a boss configured to be cooperatively received by the internal aperture.

20. The knee brace of claim 10, wherein the knee brace comprises a lock for releasably securing the handle with the linkage in a secured position.
